(19) United States
(12) Patent Application Publication
(10) Pub. No.: US 2025/0002896 A1
(43) Pub. Date: Jan. 2, 2025

(54) METHODS AND DEVICES FOR SAMPLING BODILY FLUIDS

(71) Applicant: AMA Biotech, Temple, TX (US)

(72) Inventors: Mia Mihailovic, Austin, TX (US); Paul Amador, Austin, TX (US); Samuel Mihelic, Austin, TX (US)

(73) Assignee: AMA Biotech, Temple, TX (US)

(21) Appl. No.: 18/694,956

(22) PCT Filed: Sep. 23, 2022
(86) PCT No.: PCT/US22/44601

§ 371 Date: Mar. 22, 2024

Related U.S. Application Data
(60) Provisional application No. 63/247,800, filed on Sep. 23, 2021.

(51) Int. Cl. C12N 15/10; B01L 3/00; C12Q 1/6806

(52) U.S. Cl. CPC C12N 15/1006; B01L 3/5029; C12Q 1/6806

(57) ABSTRACT

In an embodiment, the present disclosure pertains to a method of detecting specific sequences of cell-free DNA (cfDNA) from a biological sample. In an additional embodiment, the present disclosure pertains to a dipstick for capturing cfDNA from a biological sample. In a further embodiment, the present disclosure pertains to a method for at-home isolation of cfDNA from raw biological fluids. In another embodiment, the present disclosure pertains to a method for identifying optimal diagnostic toehold-mediated DNA strand displacement (TMSD) probing sites for sequence detection. In other embodiments, the present disclosure pertains to a system for visual detection of microbial cfDNA from raw biological fluids.

### CROSS-REFERENCE TO RELATED APPLICATIONS

This patent application claims priority from, and incorporates by reference the entire disclosure of, U.S. Provisional Application 63/247,800 filed on Sep. 23, 2021.

### TECHNICAL FIELD

The present disclosure relates generally to bodily fluids and more particularly, but not by way of limitation, to methods and devices for sampling bodily fluids.

### BACKGROUND

This section provides background information to facilitate a better understanding of the various aspects of the disclosure. It should be understood that the statements in this section of this document are to be read in this light, and not as admissions of prior art.

The traditional and current microbial and/or infection diagnostics landscape relies primarily on cellular DNA extraction from biosamples (e.g., blood, saliva, or swabs) prior to molecular detection of target DNA. More recently, microbial cell-free DNA (cfDNA) isolation from blood has enabled clinical diagnostics for sepsis and even organ-localized infections (e.g., tuberculosis) from plasma. To date, however, the diagnostic use of cfDNA from biosamples relies on laboratory processing (e.g., centrifugation, separation, and specialized storage). Therefore, improvements in systems and methods for isolating cfDNA from biosamples are still required for the use of cfDNA-detecting diagnostic systems and sequencing applications in an at-home environment (i.e., completely outside of the clinical setting). Silica-based solid phase extraction alternatives for highly toxic chaotropic salt solutions (e.g., guanidinium thiocyanate) have been investigated. In this research, high ionic strength accompanied by low pH (5-6) was found to facilitate binding and recovery of purified DNA from silica beads; these liquid solutions are safe for at-home use.

However, this technology is designed for commercially-available (pre-processed) DNA, but has not been demonstrated for use with raw biosamples. Furthermore, this technology relies, to some degree, on laboratory equipment, such as, for example, centrifuges. Therefore, improvements in devices, systems, and methods are required for adsorption-based DNA isolation in the at-home environment (i.e., completely outside of the clinical setting). Diagnostics that detect the presence of specific DNA sequences have remained the gold standard laboratory test for a number of decades due to their modularity and high-specificity. However, amplification-based DNA-detecting methods (e.g., polymerase chain reaction (PCR) or loop-mediated isothermal amplification (LAMP) procedures) and newer methods that have been utilized to detect infection from plasma cfDNA (e.g., next-generation sequencing (NGS)) require temperature-sensitive enzymes and, often, specialized lab equipment. Alternatively, DNA circuits sense and respond to the presence of specific nucleic acid sequences without reliance on enzymes, instead relying on toehold-mediated DNA strand displacement (TMSD) for signal amplification. Furthermore, this enzyme-free amplification strategy has been previously coupled to visual signal outputs (e.g., colorimetry or fluorimetry) that are ideal for at-home diagnostics. However, these techniques are susceptible to aberrant initiation by non-target molecules and, for this reason, have only been used with highly purified samples. Likely, this is the reason that this technology has never been demonstrated for diagnostic detection outside of the laboratory setting. As such, improvements in the devices, systems, and methods for detecting DNA fragments from complex and/or noisy biological samples using DNA circuit technology are still required.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In an embodiment, the present disclosure pertains to a method of detecting specific sequences of cell-free DNA (cfDNA) from a biological sample. In general, the method includes the following steps of: (1) placing the biological sample into a first receptacle having a first solution, where the first receptacle includes a first cap; (2) facilitating resuspension of the biological sample in the first solution; (3) replacing the first cap with a second cap, where the second cap can optionally have a dipstick; (4) mixing the first solution in the first receptacle to facilitate adsorption of extracellular DNA from the biological sample and the first solution; (5) removing the second cap from the first receptacle; (6) attaching the second cap to a second receptacle having a second solution, where the dipstick contacts at least a portion of a second solution in the second receptacle; (7) mixing the second solution in the second receptacle to facilitate saturation of the dipstick with the second solution; (8) removing the dipstick from the second receptacle; (9) placing at least a portion of the second solution from the dipstick onto a third receptacle having a third solution; (10) eluting and resuspending cfDNA from the biological sample to form a third solution; (11) transferring the third solution from the third receptacle to a reaction site, where the third solution has the cfDNA from the biological sample; and (12) detecting the cfDNA from the biological sample at the reaction site.

In some embodiments, the first solution promotes adsorption of nucleic acids to an absorbent. In some embodiments, the absorbent includes, without limitation, silica, sand, materials with similar absorbent properties of silica or sand, and combinations thereof. In some embodiments, the mixing the first solution in the first receptacle is conducted over a period of time between 30 seconds to 5 minutes. In some embodiments, the facilitating resuspension of the biological sample in the first solution is conducted over a period of time between 30 seconds to 5 minutes. In some embodiments, the second solution is a washing solution. In some embodiments, the washing solution can include, without limitation, ethanol or a solvent that is similarly miscible in water and exhibits a low nucleic acid solubility. In some embodiments, the mixing the second solution in the second receptacle is conducted over a period of time between 30 seconds to 5 minutes. In some embodiments, the method further includes the step of (13) drying the dipstick, where the drying is conducted via air drying and over a period of time between 30 seconds to 5 minutes. In some embodiments, the third solution is an aqueous solution that is more polar than a coating on the dipstick. In some embodiments, the coating is an absorbent. In some embodiments, the absorbent includes, without limitation, silica, sand, materials with similar absorbent properties of silica or sand, and combinations thereof. In some embodiments, the aqueous solution is water. In some embodiments, the eluting and resuspending is conducted over a period of time between 30 seconds to 10 minutes. In some embodiments, the third solution is transferred into at least one of reaction wells, vessels, or microfluidic chambers to permit detection of target cfDNA by at least one of complementary DNA oligos or “DNA probes” partially or completely base-pairing to a specific cfDNA sequence unique to a target organism, specimen, or gene. In some embodiments, the third solution is transferred into at least one of reaction wells, vessels, or microfluidic chambers to permit detection of target cfDNA by at least one of polymerase chain reaction (PCR), loop-mediated isothermal amplification (LAMP), or antibody-based methods unique to a target organism or specimen or gene.

In an additional embodiment, the present disclosure pertains to a dipstick for capturing cfDNA from a biological sample. In some embodiments, the dipstick includes a rod having an absorbent and a cap connected to the rod.

In some embodiments, the absorbent is composed of silica gel beads contained by a permeable mesh. In some embodiments, the absorbent is composed of a solid rod of silica. In some embodiments, absorbent can include, without limitation, silica, sand, materials with similar absorbent properties of silica or sand, and combinations thereof. In some embodiments, the rod is a tube. In some embodiments, the cap is a screw cap. In some embodiments, the rod has a length designed to fit within a 50 mL conical tube.

In a further embodiment, the present disclosure pertains to a method for at-home isolation of cfDNA from raw biological fluids. In general, the method includes the steps of: (1) adding a raw biological fluid sample to an acidic, ionic formulation to form a solution; (2) filtering clots and particulates from the solution using syringe filters; (3) binding DNA within the solution to an absorbent material via introduction of a silica material; (4) washing the DNA of the absorbent material to remove contaminants; (5) submerging the absorbent material in a fluid; and (6) eluting the DNA from the absorbent material upon drying.

In some embodiments, the washing includes washing the absorbent material with ethanol and the contaminants are at least one of proteins or lipids. In some embodiments, the fluid is water. In some embodiments, the adding a raw biological fluid sample to an acidic, ionic formulation is conducted over a predetermined time less than 30 minutes. In some embodiments, the syringe filters have a pore size of at least 0.22 μm. In some embodiments, the raw biological sample is menstrual fluid. In some embodiments, the menstrual fluid is added in a form that can include, without limitation, a saturated tampon, sanitary napkin, or menstrual fluid poured from a menstrual cup. In some embodiments, the absorbent material is contained within a syringe membrane and the DNA is adsorbed to, washed, and eluted from the membrane via syringe filtering. In some embodiments, the absorbent material is composed of a solid silica rod for a teabag application. In some embodiments, the silica rod is attached to a lid for ease of transfer. In some embodiments, the absorbent material is composed of silica beads contained inside of a netting, and surrounded by a wire mesh, for a teabag application. In some embodiments, the wire mesh is removed prior to the washing. In some embodiments, absorbent material can include, without limitation, silica, sand, materials with similar absorbent properties of silica or sand, and combinations thereof.

In another embodiment, the present disclosure pertains to a method for identifying optimal diagnostic toehold-mediated DNA strand displacement (TMSD) probing sites for microbial detection. In general, the method includes the steps of: (1) identifying a cfDNA degradome (i.e., a modeling degradation pattern to predict specific resulting cfDNA fragments in a given microbiome) or degradation pattern of cfDNA of a targeted microbe; (2) compiling a library of potential target toehold fragments chosen as toeholds represented in the degradome or the degradation pattern; (3) designing a corresponding library of target sequences having sequence extensions from each toehold to fill a design space of a predetermined length range for TMSD circuits; and (4) ranking of the corresponding library of target sequences based, at least in part, on at least one of toehold abundance, length of target sequence, uniqueness of target sequence, or predicted energetics of interaction between the target sequences and diagnostic DNA molecules.

In some embodiments, the degradome or the degradation pattern includes modeled degradation patterns to predict specific resulting cfDNA fragments in a given microbiome. In some embodiments, the degradome or the degradation pattern of a microbial cfDNA of interest is determined by activity nucleases of species, including and outside of a species of interest, represented in a biosample-corresponding microbiome. In some embodiments, the degradome or the degradation pattern is identified computationally via identification of endonucleases in a biosample of interest via homology-based alignment and corresponding mapping of expected nuclease patterns. In some embodiments, the degradome or the degradation pattern is identified via experimental metagenomic DNA sequencing using specialized next-generation sequencing (NGS) preparation methods, and where DNA size-selected fragments are not fragmented prior to sequencing. In some embodiments, the degradome or the degradation pattern is compiled computationally via identification of endonucleases in a biosample of interest via homology-based alignment and corresponding mapping of expected nuclease patterns and via experimental metagenomic DNA sequencing using specialized NGS preparation methods, and DNA size-selected fragments are not fragmented prior to sequencing. In some embodiments, standard probabilities of restriction enzyme activity to create unique toeholds is obtained from comparing mapped sequence positions corresponding to fragment ends to those identified by at least one of computational methods or experimental methods. In some embodiments, only toehold lengths greater than 4 nucleotides are considered. In some embodiments, target sequence libraries are based, at least in part, on target sequence lengths between 25 and 40 nucleotides in length. In some embodiments, machine learning approaches are used to optimize the ranking using features, including, but not limited to, length of probes, target overhang length, or cleavage likelihood (based, at least in part, on empirical degradome sequencing).

In other embodiments, the present disclosure pertains to a system for visual detection of microbial cfDNA from raw biological fluids. In some embodiments, the system generally includes: (1) an acidic, ionic solution configured for mixing with raw a biosample; (2) an absorbent material configured for extracting biomolecules from the solution; (4) a washing solution configured for washing the absorbent material of non-nucleic acid biomolecules; and (5) an elution solution comprising diagnostic nucleic acid molecules for strand displacement, where the system is configured to provide a positive visual output if a presence of a diagnostic target is detected.

In some embodiments, the elution solution enables PCR-based, LAMP-based, or DNA-antibody-based therapeutics. In some embodiments, the elution solution enables a TMSD cascade. In some embodiments, targets are informed via a method to select optimal targets that incorporates at least one of degradome information, thermodynamics, or homology analysis. In some embodiments, the absorbent material is a silica-based material. In some embodiments, the absorbent material can include, without limitation, silica, sand, materials with similar absorbent properties of silica or sand, and combinations thereof. In some embodiments, TMSD optimization is achieved using at least one of established DNA circuit-modeling software or logic multiplexing. In some embodiments, TMSD optimization is achieved by destabilizing double-stranded DNA (dsDNA) fragments having the diagnostic DNA target sequence using a molar excess of oligonucleotides directed towards a surrounding sequence space (termed “pry ‘n’ probe” herein). In some embodiments, colorimetric signal is enabled by color catalysis of a colorless substrate upon displacement-induced formation of a DNAzyme. In some embodiments, fluorometric signal is enabled by TMSD cascade-induced separation of fluorophore and quencher. In some embodiments, the system further includes a distinct device for human sight-interpretation of fluorometric signal. In some embodiments, the raw biosample is menstrual fluid, and microbes to be detected are causative agents of sexually transmitted diseases or infections. In some embodiments, the visual output is a digital display relaying the results of a digital measurement of the biological sample. In some embodiments, the digital measurement is optical density enabled by color catalysis of a colorless substrate upon displacement-induced formation of a DNAzyme. In some embodiments, the optical density is measured with an optical device having a light sensor, a light source that includes one or more absorbance peaks of the catalyzed color, and a bandpass filter specific to one or more absorbance peaks of the catalyzed color. In some embodiments, the digital measurement is fluorescent intensity enabled by TMSD cascade-induced separation of fluorophore and quencher. In some embodiments, the intensity is measured with an optical device having a light sensor, a light source that includes the excitation frequency of the fluorophore, one bandpass filter specific to the fluorophore emission band, and a bandpass filter specific to the fluorophore excitation band. In some embodiments, the digital measurement is electrical conductance enabled by TMSD cascade-induced conductivity change. In some embodiments, the raw biosample is menstrual fluid. In some embodiments, genetic markers of disease states are detected. In some embodiments, the genetic markers of disease states comprise genetic mutations or genes from other organisms living inside the body indicating cancer, heart disease, infertility, and combinations thereof. In some embodiments, the raw biosample is puss from a wound or a blood draw sample, and wherein genetic markers of antimicrobial resistance are detected.

In additional embodiments, the present disclosure pertains to method of detecting specific sequences of cfDNA from a biological sample. In general, the method includes: (1) placing the biological sample into a first receptacle having a first solution, where the first receptacle has a first contact surface; (2) facilitating resuspension of the biological sample in the first solution; (3) replacing the first contact surface with a second contact surface; (4) mixing the first solution containing the biological sample in the first receptacle to facilitate adsorption of extracellular DNA from the biological sample and the first solution; (5) removing the second contact surface from the first receptacle; (6) attaching the second contact surface to a second receptacle having a second solution, where the second contact surface contacts at least a portion of a second solution in the second receptacle; (7) mixing the second solution in the second receptacle to facilitate saturation of the second contact surface with the second solution; (8) removing the second contact surface from the second receptacle; (9) placing at least a portion of the second solution onto a third receptacle having a third solution; (10) eluting and resuspending cfDNA from the biological sample to form a third solution; (11) transferring the third solution from the third receptacle to a reaction site, where the third solution includes the cfDNA from the biological sample; and (12) detecting the cfDNA from the biological sample at the reaction site.

In some embodiments, the first solution promotes adsorption of nucleic acids to an absorbent. In some embodiments, the absorbent includes, without limitation, silica, sand, materials with similar absorbent properties of silica or sand, and combinations thereof. In some embodiments, the mixing the first solution in the first receptacle is conducted over a period of time between 30 seconds to 5 minutes. In some embodiments, the facilitating resuspension of the biological sample in the first solution is conducted over a period of time between 30 seconds to 5 minutes. In some embodiments, the second solution includes a washing solution. In some embodiments, the washing solution can include, without limitation, ethanol and a solvent that is similarly miscible in water and exhibits a low nucleic acid solubility. In some embodiments, the mixing the second solution in the second receptacle is conducted over a period of time between 30 seconds to 5 minutes. In some embodiments, the second contact surface includes a dipstick. In some embodiments, the method further includes the steps of drying the dipstick, where the drying is conducted via air drying and over a period of time between 30 seconds to 5 minutes. In some embodiments, the third solution an aqueous solution that is more polar than a coating on the dipstick. In some embodiments, the coating is an absorbent. In some embodiments, the absorbent can include, without limitation, silica, sand, materials with similar absorbent properties of silica or sand, and combinations thereof. In some embodiments, the aqueous solution is water. In some embodiments, the eluting and resuspending is conducted over a period of time between 30 seconds to 10 minutes. In some embodiments, the third solution is transferred into at least one of reaction wells, vessels, or microfluidic chambers to permit detection of target cfDNA by at least one complementary DNA oligos or “DNA probes” partially or completely base-pairing to a specific cfDNA sequence unique to a target organism or specimen or gene. In some embodiments, the third solution is transferred into at least one of reaction wells, vessels, or microfluidic chambers to permit detection of target cfDNA by at least one of polymerase chain reaction (PCR), loop-mediated isothermal amplification (LAMP), or antibody-based methods unique to a target organism or specimen or gene.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIG. 1 illustrates a testing method according to aspects of the present disclosure.

FIGS. 2A-2C illustrate menstrual fluid cell-free DNA (cfDNA) isolation. FIG. 2A shows a cartoon depiction of noisy menses environment (left), particle filtered menses (middle), and DNA adhesion to silica beads (right). The menses environment contains lysed cells and nucleases (“Pacman”-shape) degrading cfDNA (double helices). The minimal processing to isolate DNA includes filtering and DNA-silica adhesion (beads in dashed line). FIG. 2B shows absorption spectra of crude menses sample, minimally processed cfDNA isolate, and further-processed cfDNA isolate using a DNA clean-up kit. The crude menses (suspended in a buffer) has high signal that obscures the DNA peak (˜260 nm); the minimally processed cfDNA isolate has some protein present (˜280 nm), while the DNA clean-up kit is further purified, confirming the abundance of cfDNA in the “cfDNA isolate” sample. FIG. 2C shows absorption spectra of a syringe-based purification (Method 1) and “dipstick” cfDNA isolation protocols (Method 2). Method 1 has higher absorbance at 280 nm and 410 nm than Method 2, indicating more protein and hemoglobin contamination, respectively.

FIG. 3 illustrates a top-to-bottom flow of a cfDNA isolation (Method 1) from raw menses (syringes required).

FIG. 4 illustrates an improved user-friendliness top-to-bottom flow of cfDNA isolation (Method 2) from raw menses (syringes not required).

FIGS. 5A-5C illustrate menstrual cfDNA degradome. FIG. 5A shows bioinformatic prediction of endonuclease cut-sites are performed using public databases and sequence alignment tools. FIG. 5B shows that reversed-phase high-performance liquid chromatography (RP-HPLC) reveals that the cfDNA isolates (raw samples stored for one week at room temperature (RT) and 4° C., respectively) contain a significant portion of DNA fragments below 1 kbp in length (first peak in 100 bp ladder, ˜14.6 minutes retention time). Length distribution shifted to lower lengths (especially between 100 and 500 bp, retention times ˜9 min and 13.8 min, respectively) when the menses sample is incubated RT vs. fridge temperature (4° C.) for a week. FIG. 5C shows next-generation sequencing (NGS) of menses cfDNA isolate used to determine the probability of fragmentation at putative cut-sites.

FIG. 6 illustrates a specific proposed use of bioinformatic target selection pipeline. The total target design space is the virulence genes associated with the microbe of interest: (1) target design space is searched for cut sites identified in vaginal genomes; (2) cut sites are scored on their ability to be probed, assumed to be proportional to their abundance and degree of fragmentation (inverse length of fragment); (3) candidate probes are cross-referenced for uniqueness in the vaginal microbiome-if not unique at the current length, the probe length may be extended; (4) candidate probes are further scored on their predicted energies of hybridization to the toehold; and (5) candidate probes with the best score are experimentally tested.

FIG. 7 illustrates mock instructions for microbial-detecting systems for use with raw menstrual fluid biosample. In the first step, a menstrual sample is added to Solution 1, and shaken for 2 min. In the second step, the cap from Solution 1 is replaced with a dipstick cap, and shaken. In the third step, the dipstick cap is replaced onto Solution 2, and shaken. The third step is then repeated for Solution 3 (step four). In step five, the dipstick cap is removed, a designated number of drops of Solution 4 are added, and a designated amount of time is waited. In step six, a colorimetric result is read to indicate microbial detection.

FIGS. 8A-8C illustrate application of DNA strand displacement (DSD) circuits to cfDNA isolate. FIG. 8A shows a simple target amplification DSD cascade for preliminary application to noisy biosamples. FIG. 8B shows a colorimetric time-course readout from the DSD cascade applied to water with (“5 nM target”) and without (no target) the target sequence and a minimally-processed cfDNA isolate sample with (“cfDNA isolate+5 nm target”) and without (“cfDNA isolate”) the target sequence. The baseline absorption of the cfDNA isolate alone without the colorimetric DSD cascade is shown as “cfDNA isolate, no DSD”.

FIGS. 9A-9B illustrate various dipsticks with a silica rod according to aspects of the present disclosure.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

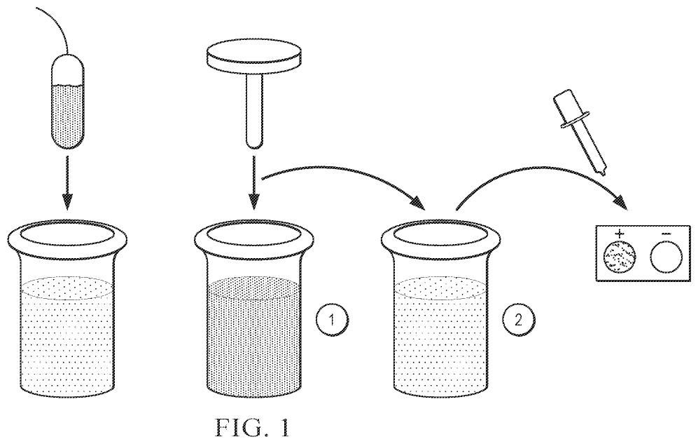

In some embodiments, the present disclosure pertains to at-home isolation of cell-free DNA (cfDNA) from user-collected biosamples. FIG. 1 illustrates a system leveraging the devices, systems, and methods of the present disclosure. FIG. 1 shows an overview of a completely at-home biosample method and device.

Embodiment 1. In order to expand the scope of current at-home diagnostics to highly accurate DNA detection, protocols to safely and effectively isolate cfDNA from user-collected biosamples are important. Such biosamples can include, without limitation, blood, saliva, urine, wound exudate, pus, genital discharge, and menstrual fluid (menses).

Embodiment 2. In some embodiments, the biosample is menstrual fluid. As such, disclosed herein are two methods for at-home isolation of cfDNA from menstrual fluid that do not require laboratory equipment (e.g., centrifuges) or toxic chemicals. These methods will work on all aforementioned biosamples, though not limited to those biosamples listed. This is evidenced by data presented herein based on menses biosamples. In some embodiments, these methods can be used with other menses-capturing commercial products, including menses-saturated sanitary napkins, or menstrual fluid cup samples, in a similar manner, so long as the menstrual fluid sample is transferred into a container for further processing.

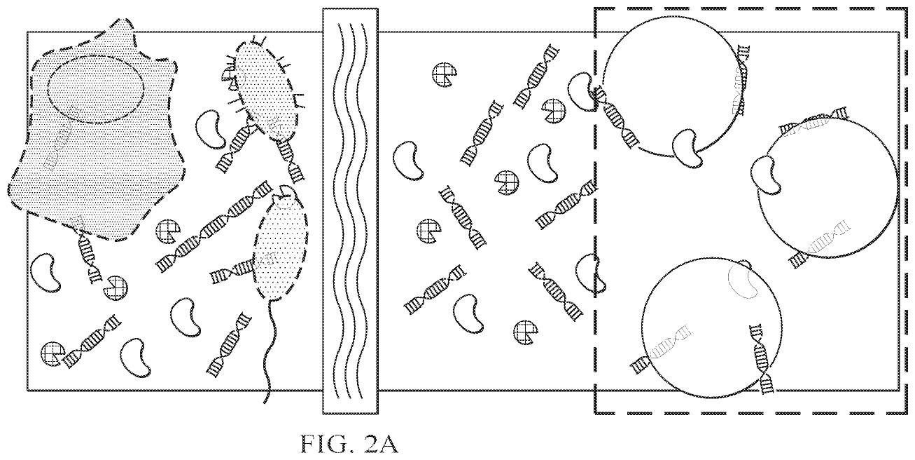

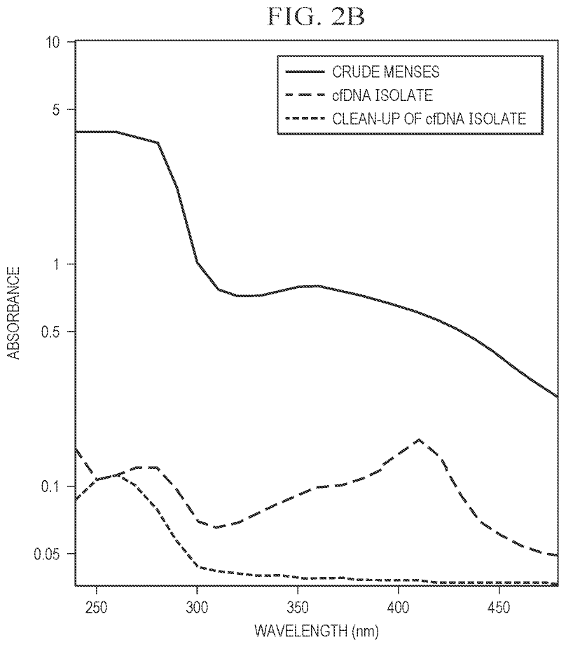

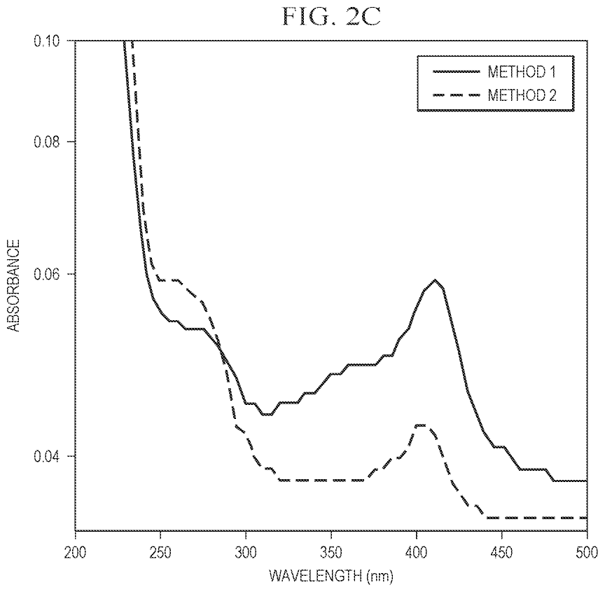

The protocols as disclosed herein are based, at least in part, on research that investigated silica-based solid phase extraction alternatives for highly toxic chaotropic salts (e.g., guanidinium thiocyanate) for the purpose of DNA re-capture (FIG. 2A). Notably, in this research, high ionic strength accompanied by low pH (5-6) was found to facilitate DNA binding and recovery from silica beads. These liquid solutions are safe for at-home use, and have frequently been used at home. In some embodiments, silica-based materials, in general, can be replaced by any absorbent material. For example, in some embodiments, the absorbent materials utilized herein can include, without limitation, silica, sand, absorbent materials that have similar absorbent properties of silica or sand, and any combination of the same and like. As such, absorbents and absorbent materials of the present disclosure can include any materials or solutions that assist in, or facilitate, DNA adsorption.

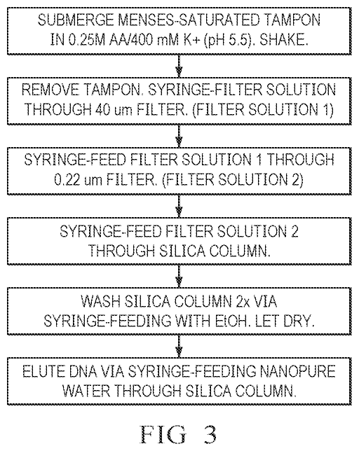

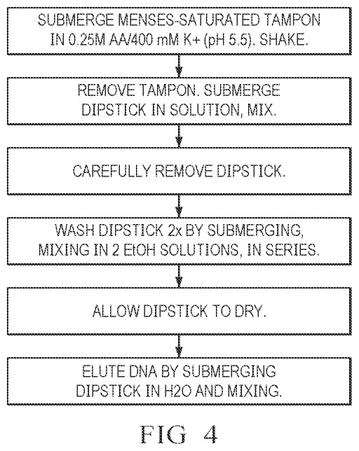

The first protocol requires the repeated (in series) transfer of solutions to syringe filters with varying filter size or membrane composition, enabling an average recovery of 20-40 μg of cfDNA (absorbance peak ˜260 nm) per menses-saturated tampon/menstrual cup with some protein (hemoglobin absorbance peak ˜410 nm) impurities (FIG. 2B, FIG. 2C, and FIG. 3). The second protocol is more user-friendly, utilizing a silica gel dipstick that is submerged in a variety of free-standing solutions (referred to herein as a “dipstick”) to capture cfDNA, and enabling recovery of 50-70 μg of cfDNA per menses-saturated tampon, with enhanced removal/separation of proteins than method 1 (FIG. 2C and FIG. 4). Both methods are described in more detail below.

Briefly, the first method for cfDNA isolation from a saturated tampon begins with tampon submergence and shaking in 25 mL of 0.25 M acetic acid (AA) containing 400 mM K⁺ (adjusted to pH 5.5), followed by incubation at room temperature (RT) for at least 30 minutes. Next, the tampon is removed, and the corresponding menses solution is syringe-filtered in two stages (40 μm and 0.22 μm) prior to a slow capture through a syringe-fed silica column. This silica column is washed twice with 10 mL ethanol for removal of non-nucleic acid biomolecules, and once dry, DNA is eluted by syringe-flowing 1 mL nano-pure water through the membrane. FIG. 3 illustrates a flow chart of this process. The development of this protocol has provided minimally-processed samples, with reduced content of non-DNA biomolecules, including, but not limited to, proteins and lipids. However, this processing protocol is not capable of complete removal of impurities (e.g., proteins, aggregates, and particles) as evaluated via spectrophotometry (FIG. 2B, cfDNA Isolate).

The second method for cfDNA isolation from raw biosamples relies largely on the utility of a silica-gel bead core, held together by a mesh netting (for larger-scale filtration of biosample debris), as stated earlier, referred to as a “dipstick”, that is used for DNA adsorption. Alternatively, a solid silica gel rod can be utilized as a dipstick style probe. This dipstick, in some embodiments, can be attached to a screw-cap lid for ease of transfer and immersion through various tubes and/or solutions, referred to as a “dipstick cap”. Similar to the first method, this method begins with tampon submergence and shaking in 25 mL of 0.25 M acetic acid containing 400 mM K⁺ (adjusted to pH 5.5), followed by incubation at RT for at least 30 minutes. Next, the tampon is removed from the solution, and the dipstick is inserted and vigorously mixed for adsorption of biomolecules. Afterward, the dipstick is washed 2× by submerging and mixing in tubes containing ethanol (EtOH). The dipstick is then removed, allowed to dry, and then submerged in water to elute adsorbed DNA. FIG. 4 illustrates a flow chart of this process.

Embodiment 3. In some embodiments, the present disclosure relates to a bioinformatic approach, based on biosample microbial biodiversity and associated DNA processing, to identify optimal cfDNA target fragments in biosamples. cfDNA is an ideal diagnostic target for at-home utility due to its fragmented condition that offers high accessibility (as compared to highly-compact chromosomal DNA). For example, in human plasma, human and microbial cfDNA molecules are fragmented to a length of about 100 base pairs on average, thousands of times shorter than the original, intact genome (e.g., 1 Mbp Chlamydia chromosome). It is believed that this fragmentation is a result of nucleases, or DNA-cleaving enzymes, that are released during the apoptotic process. Although only end-targeting exonuclease DNase II has been implicated in plasma samples to date, it is likely that many more nucleases will be active in biosamples in which a high diversity of microbes are present, which can include, but are not limited to, saliva, wound exudate, pus, genital discharge, menstrual fluid, and combinations of the same and like. These microbes produce a particular nuclease of interest, endonucleases, some of which can degrade DNA in a predictable manner, cleaving double-stranded DNA at specific sequences called restriction sites.

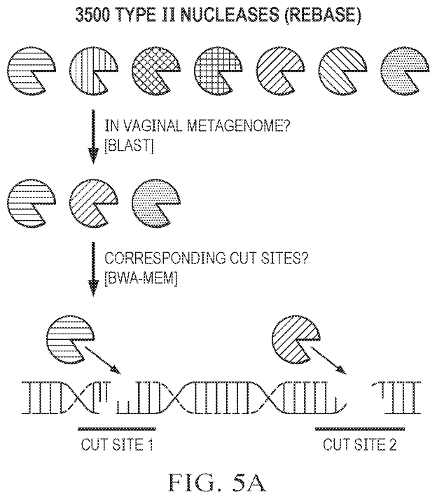

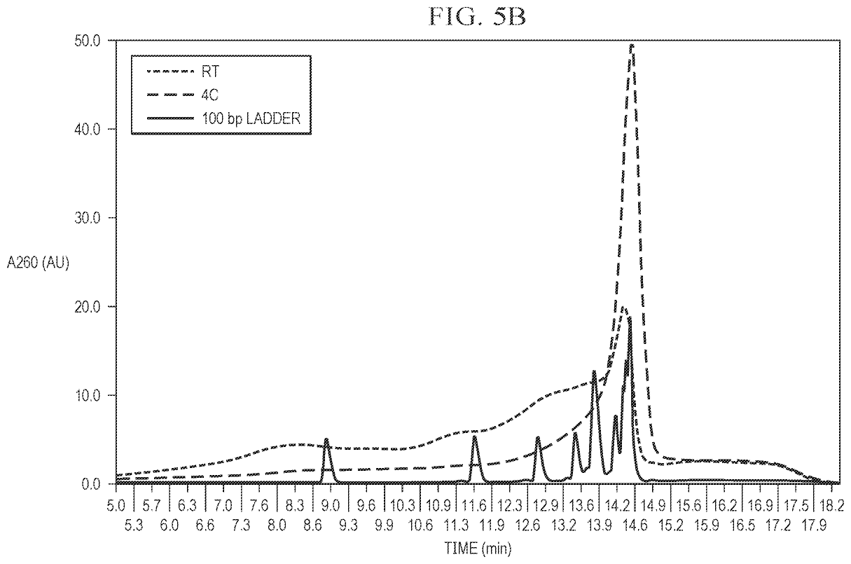

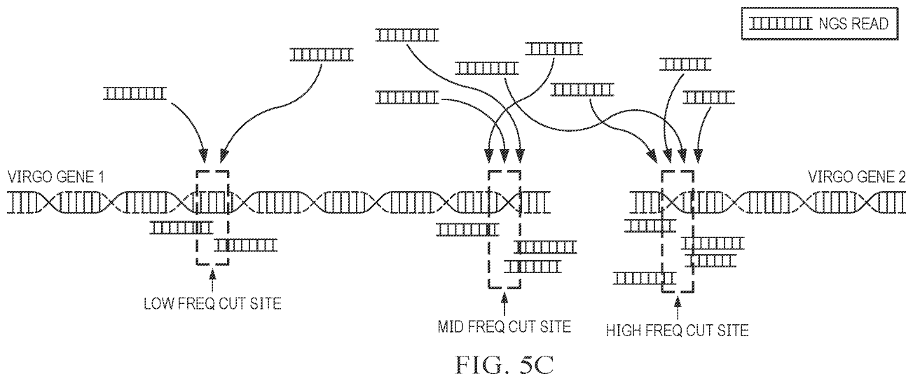

Endonucleases often cleave double-stranded DNA in a staggered manner that leaves short single-stranded overhangs (4-7 nucleobases). Such short sequences of single-stranded DNA, or “toeholds”, would be appealing diagnostic probing targets because they are highly exposed and predictable by sequence identification (FIG. 5A). Although the exact mechanisms of cfDNA processing in menses are unknown, bioinformatic analyses performed, namely a basic local alignment search tool (BLAST) search for homologs of >53,000 accepted Type II nucleases that produce single-stranded overhangs (REBASE, a database for DNA restriction enzymes) against the 2.8 Bbp Human Vaginal Non-redundant Gene Catalog (VIRGO), suggest that >13,500 Type II nucleases are represented within the vaginal environment, as defined by >50% of the coding sequence with >70% sequence identity. However, cfDNA degradation may not be as widespread as suggested bioinformatically due to for a number of reasons, including, but not limited to: (i) proteins may be occluding certain sequence-specific cut sites via intermolecular binding; or (ii) nucleases without sequence targeting specificity (e.g., exonucleases) may predominate. Furthermore, cfDNA processing patterns will likely vary person-to-person due to differences in vaginal microbiome composition.

To experimentally observe the specific fragmented products, that may or may not be products of endonucleases (e.g., products degraded by enzymes with less rational targeting) in biosamples, next-generation sequencing (NGS) can be performed on cfDNA. Given that shorter double-stranded DNA (dsDNA) fragments are more amenable to strand separation via diagnostic molecules, fragments less than 300 nucleotides in length, concentrated via column-based size-exclusion, are analyzed. A NEBNEXT® ULTRA™ II DNA Library Prep Kit is used prior to paired-end sequencing analysis on an Illumina MiSeq instrument. Importantly, traditional library fragmentation of size-excluded cfDNA is not performed in order to preserve the native fragments and enable the recognition of overhang-containing ends. NGS reads will be aligned to the VIRGO genome via Burrows-Wheeler alignment (BWA)-mem. Mapped sequence positions corresponding to fragment ends are compared to those identified in the bioinformatic approach to obtain standard probabilities of restriction enzyme activity. Single-stranded DNA sequencing is additionally performed via established library preparation methods to identify cfDNA nicks. Nicks are DNA backbone cuts which occur only on one strand of double-stranded DNA (dsDNA) that have shown high frequency in microbial cfDNA of the plasma (˜1-2 per 200 bp fragments on average). These sites provide another point of weakness in natural dsDNA to enable targeting. In all, this two-tier approach will identify highly available toeholds for use in establishing first-contact with diagnostic DNA molecules.

Once the empirical degradome are elucidated, a library of candidate target sequences is selected as genomic regions (within virulence and resistance genes) proximal to fragment ends (as identified via degradome investigation), which may or may not correspond; however, genomic regions corresponding to endonuclease cut sites producing, for example, but not limited to, a 4-nucleotide minimum overhang would be ideal, given that this optimal length of overhang for toehold-mediated DNA strand displacement (TMSD) has been kinetically characterized as at least 4 nucleotides. Each cut site yields two flanking overhangs and thus two opportunities to initiate a strand displacement reaction. However, given the susceptibility to exonucleases by synthetic hairpin reactants containing 3′ toeholds (and thus specificity of targeting), 3′ overhangs will serve as potential initiation points on the target sequence.

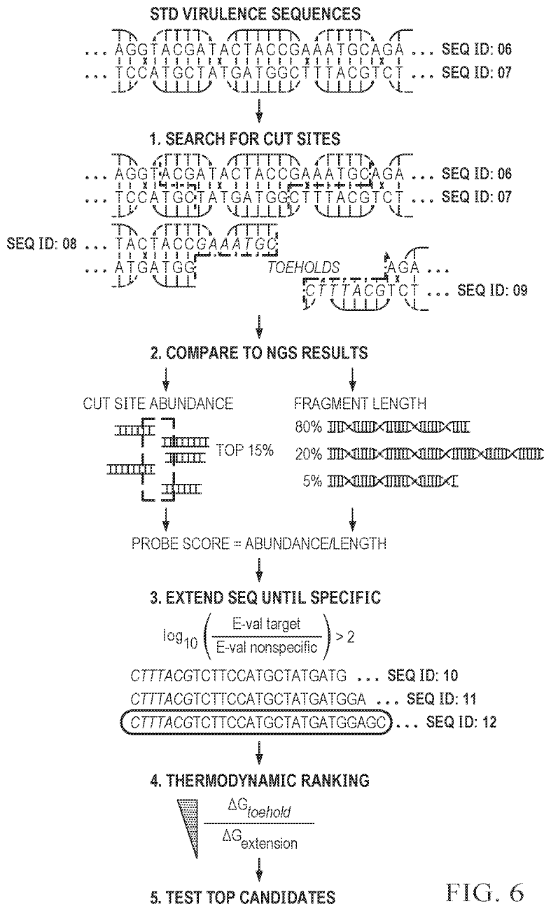

These candidate toehold sites are ranked by an interaction parameter representing toehold abundance and likely cfDNA fragment length (both probabilistically determined from NGS data) corresponding to the exposed single-stranded overhangs they create (FIG. 6, steps 1 and 2). Next, several corresponding unique pathogenic DNA targets are chosen, based on toehold candidates, to fill the design space of a predetermined length range for TMSD circuits (e.g., 25-40 nucleotides). Candidates from this subset are screened against VIRGO using BLAST to determine pathogen uniqueness (FIG. 6, steps 3 through 5) for purposes of a specificity-based reranking step. In the last re-ranking step, candidates with favorable, that is, “most-displaceable” energetics (high free energy ratio of target toehold:remainder of sequence) are identified. Top ranking candidates are tested in a plate-based assay against infected (and non-infected) samples in order to assign corresponding performance metrics. To further increase sensitivity, the weighting of parameter features already represented in a bioinformatic pipeline can be optimized using experimental results (e.g., abundance, length, guanine-cytosine (GC) content, energetics, etc.) via machine learning methods.

As part of an effort to confirm and characterize the fragmented state of cfDNA from user-processed biological fluids, reversed-phase high performance liquid chromatography (RP-HPLC) was performed on the minimally-processed isolated menstrual cfDNA sample (obtained via the protocol of Embodiment 2, discussed above), further purified using a Zymo DNA Clean & Concentrator kit (absorption spectrum shown in FIG. 2B). This experiment revealed a broad length distribution of cfDNA with a significant portion of fragments below 2 kbp and a majority of fragments below 23 kbp (FIG. 5B). Additionally, fragmentation continuing ex vivo was confirmed by including an equivalent menstrual sample which was stored at room temperature for 1 week. The distribution of fragments shifted towards shorter lengths for this heavily-degraded sample.

In light of the confirmation of degradation of cfDNA, this embodiment details the design of a pipeline for design and ranking candidate target sequences by incorporating: (1) probabilistic models for degradation patterns of the menstrual cfDNA; (2) target sequence uniqueness to the pathogen of interest; and (3) energy release of targeting. Notably, candidate pathogenic microbe target sequences (e.g., sexually transmitted diseases, sexually transmitted infections, etc.) will be constrained to only genes known to contribute to virulence or resistance, because these genes are highly conserved and contribute to the disease state. Indeed, virulence and resistance genes are often the targets of polymerase chain reaction (PCR)-based bacterial detection methods. It is noteworthy that this is the first instance of modeling degradation patterns to predict specific resulting cfDNA fragments in a given microbiome. This concept is referred to as the degradome, and the related field of study, degradomics. The application of degradomics to the microbiome environment of the vagina for the purpose of designing TMSD circuits for detecting the presence of diseases is a novel proposition.

To achieve full characterization of the cfDNA degradome of any biosample (obtaining the degradome for this sample), a two-tier approach can be taken. First, endonucleases with likely activity in the biosample of interest can be bioinformatically predicted, along with corresponding cfDNA cut sites (FIG. 4A). Secondly, experimental NGS can be performed to obtain the identities of individual fragments to confirm highest-activity nucleases in this bio-environment (FIG. 4C). This process is stochastic, and, as such, the goal of NGS is to verify the degradation patterns proposed bioinformatically that can be used to predict regions of the target genome with a higher likelihood of degradation due to specific cleaving events.

Bioinformatic prediction of cfDNA degradation patterns are facilitated by curated databases and established bioinformatic tools. For example, REBASE hosts a wealth of information, including coding sequence as well as target sequence (and associated cut patterns), for more than 3,500 Type II nucleases whose enzymatic DNA processing patterns target with sequence specificity. Similarly, recent NGS analysis is currently available for a variety of relevant bioenvironments. For example, over 200 vaginal metagenome sequences have supported the compilation of a comprehensive, non-redundant vaginal gene catalog that covers more than 95% of the human vaginal microbiome (VIRGO). These sequences, coupled with established local alignment tools together enable the identification of sequences encoding for endonucleases from the vaginal genome, as well as associated cut sites within menstrual cfDNA. Databases are searched for microbiome homologs of all known Type II endonucleases that produce single-stranded DNA overhangs (excluding those producing blunt ends without toeholds), as defined by an E-value (probability of random match) less than 0.01, representing at least 70% of the coding sequence. Next, for these biosample-homolog endonucleases, sites are located throughout VIRGO that contain corresponding recognition sites. For this task, BWA algorithms are utilized for exact matches. Notably, while this produces a library of all potential overhangs, experimentation is necessary to confirm that these sites are being cleaved and to determine their relative frequencies of cleaving to identify the most likely fragmentations.

Embodiment 4. In some embodiments, the present disclosure pertains to a platform for at-home diagnostic detection of microbial cfDNA from minimally-processed biosamples using DNA circuit technology and, for example, Embodiment 3 as disclosed herein. The device corresponding to Embodiment 4 integrates the isolation of cfDNA from raw biosample with the detection of target DNA (as designed via Embodiment 4) using TMSD. Importantly, this is the first instance of detecting target DNA, selected based on its accessibility due to natural biological processes, from user-processed biological samples using TMSD technology.

In addition, TMSD cascades can be modified to achieve desired detection accuracy specifications, given that background leakage typically threatens their diagnostic specificity. Already, the use of an established DNA circuit previously used for in vitro applications has been chartered. However, when tested on minimally-processed menses samples, increased background signals were observed (Embodiment 5, described below). Such systematic error, caused by complex biological samples, requires redesign of the DNA circuit to improve robustness to the noisy environments. Fortunately, DNA circuits are highly predictable and can even be modeled using established kinetics-based definitive screening design (DSD) design software programs. These software programs allow to computationally test design modifications (detailed in Embodiment 5 below), including reactant stability and at least one failsafe (e.g., multiple target consensus), to improve robustness, namely specificity, within complex diagnostic samples.

Briefly, this embodiment is a device incorporating: (1) cfDNA isolation reliant on silica-based materials in a variety of embodiments, including, but not limited to, a syringe filter application, a dipstick application, or integrated into a wearable tampon device; and (2) microbial DNA detection using TMSD and a variety of outputs, including, but not limited to, colorimetry (not requiring any additional detection device) or fluorimetry (requiring a separate signal converter for visual detection).

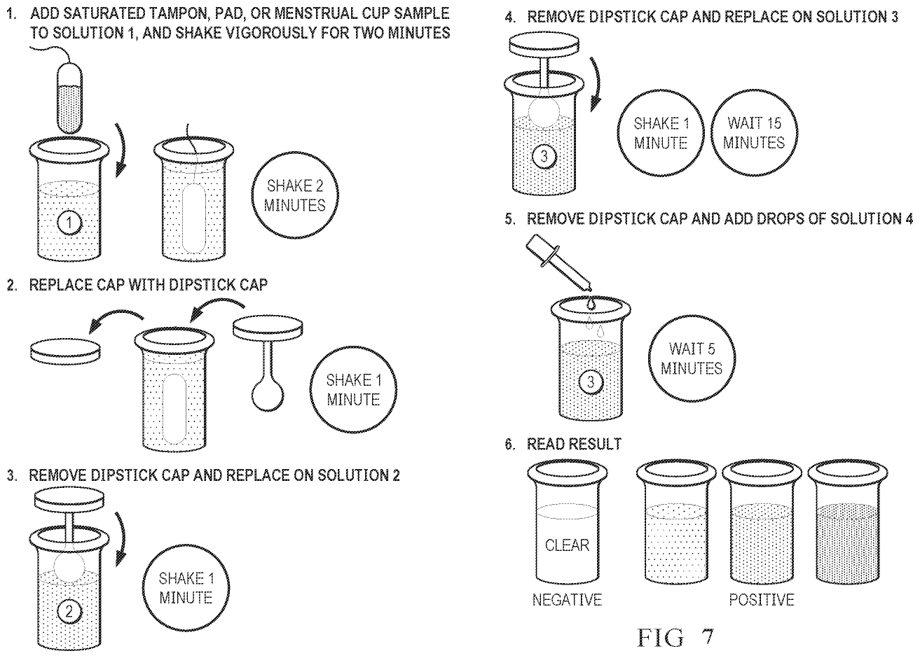

In some embodiments, the device may require large biosamples (e.g., a saturated tampon). In some embodiments, the device can be a microfluidic device in which a drop of menstrual fluid is added and processed in microfluidic chambers. In some embodiments, the systems and devices may benefit from future studies indicating molar amounts of target DNA (which likely varies per microbial pathogen under investigation) within different volumes of biosamples. Instructions for an example macrofluidic device of Embodiment 4 with use of a raw menstrual fluid biosample is illustrated in FIG. 7.

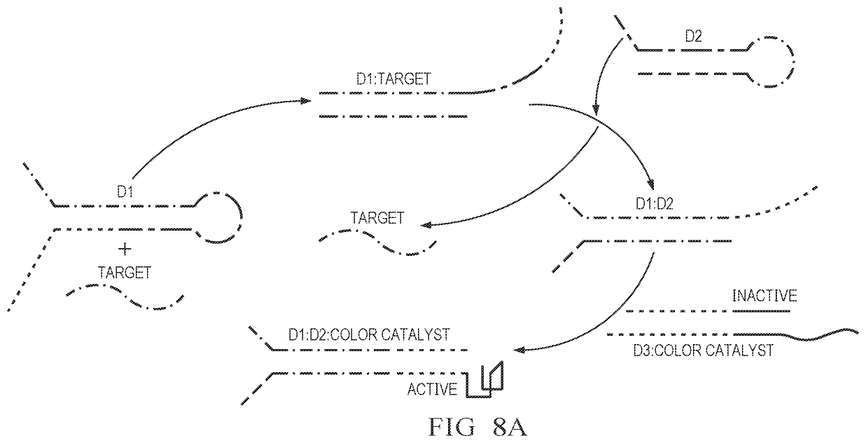

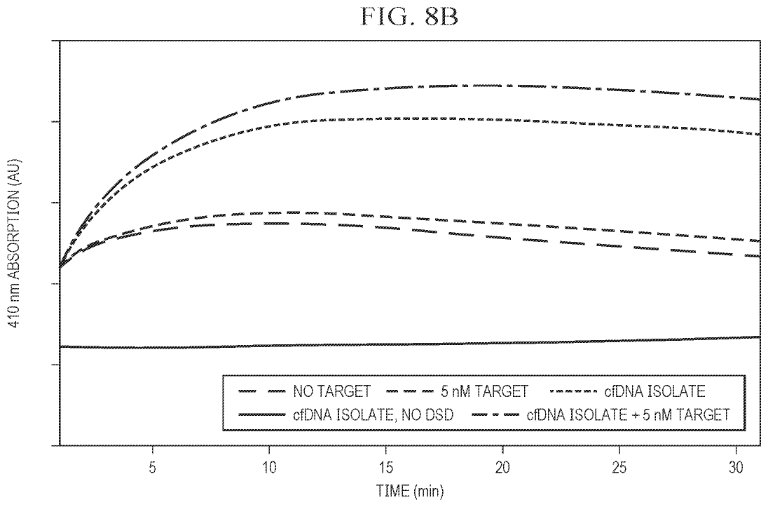

Embodiment 5. In some embodiments, the present disclosure pertains to a device, for example, the device of Embodiment 4, where the biosample is menstrual fluid (Embodiment 2) and where the foreign cfDNA of interest corresponds to sexually-transmitted pathogens. It was hypothesized that the complex menses cfDNA environment would challenge the robustness of DSD cascades compared to previous highly-controlled in vitro environments. To test this hypothesis, a simple catalytic hairpin circuit that was adapted for colorimetric output (FIG. 8A) was employed. The target DNA region had not been optimized using rules outlined in Embodiment 3.

This specific cascade design was chosen for the following reasons: (i) high sensitivity; and (ii) a modular and simple design. When these diagnostic molecules were used to target a highly-specific 30 nt region (E-value 0.03) of Actinobaculum sp. (one of 10 highly represented phyla in the vaginal microbiome, on average) within a minimally-processed cfDNA isolate sample (˜1 μM cfDNA), signals comparable to >5 nM of a positive control oligonucleotide was observed (FIG. 8B). The diagnostic molecules had the following sequences: M2: GCACTTTC GAG CATCTTCG GCCATTTC GCTATATCCTCCACG GAAATGGC CGAAGATG CTC CTGATGTG GGCTAAAG (SEQ ID: 01), A2: GCCATTTC CGTGGAGGATATAGC GAAATGGC CGAAGATG CTC GCTATATCCTCCACG (SEQ ID: 02), S2_Dzinh: CTC CTGATGTG GGCTAAAG TCCCTC (SEQ ID: 03), S2_Dz: CTGGGAGGGAGGGAGGGA CTTTAGCC CACATCAG GAG CATCTTCG (SEQ ID: 04), and C1: GAAATGGC CGAAGATG CTC GAAAGTGC (SEQ ID: 05). At this molar concentration it is likely that the 30 nt molecular target is present at femtomolar concentrations (assuming: (i) uniform abundance of all ˜600 species in the vaginal microbiome; and (ii) average microbial genome size of 2 Mbp). Thus, it is clear that menses cfDNA drastically increases background signal, hurting the specificity, and accuracy, of the simple diagnostic cascade. Importantly, these results suggest that aberrant catalytic hairpin activation is occurring, because in the absence of the initiator hairpin, little background signal was observed (FIG. 8B, cfDNA isolate, no DSD). In order to compensate for the roughly 6 orders of magnitude (nano versus femto) of background that is being observed, considerable optimization should be made to the strand-displacement circuit. Such optimization can, in part, come from the bioinformatic selection of targets detailed in Embodiment 3.

For targeting specificity not addressed by Embodiment 3, it is encouraging that DNA circuits with picomolar detection limits have been demonstrated, and those with more complex designs have been simulated to even lower (femtomolar range) concentrations (using a two-layer circular cascade). Associated studies showed the ability to achieve such low detection limits by purifying DNA substrate strands, that are subject to nucleotide-level impurities during chemical synthesis, and tempering the catalytic activity of the circuit via nucleotide mismatch. Additionally, increasing hybridization lengths can increase the stability of the reactant catalytic hairpins and improve the test specificity. Combining these design considerations can decrease background signal by orders of magnitude. These coupled optimizations can be modeled using kinetic DNA circuit software programs, for example, the Visual DNA Strand Displacement tool, to screen candidate designs for experimental testing.

If specificity has not been achieved to desired diagnostic accuracy using individual targets designed via the aforementioned approaches, the targeting of highly-specific pathogenic cfDNA fragments can be multiplexed using DSD logic gates. By multiplexing several targets with logic gates, the individual signals can be integrated and a consensus signal with higher confidence will be obtained. In particular, sensitivity can be improved with OR gate combinations and specificity with AND gates. In this way, various regions of the pathogenic genome of interest can be simultaneously targeted by multiple probes to improve the performance of the diagnostic assay.

Another contingency to increase specificity can include a method involving the addition of an excess of oligonucleotides directed towards the surrounding sequence space (e.g., upstream and/or downstream) of the diagnostic target sequence, in an attempt to destabilize the dsDNA interactions, favoring target hybridization (i.e., “pry ‘n’ probe” as termed herein). Altogether, these TMSD modifications support the improved specificity of such circuits to levels amenable for purposes of completely at-home microbial DNA detection from minimally-processed biosamples.

With respect to the restriction enzyme overhangs, for example, those in Embodiment 3, the present disclosure leverages empirical degradation data (e.g., sequencing data) to design diagnostic probes that are complementary to the regions that are most accessible for base-pairing, such as, for example, single-stranded (frayed ends of double-stranded) DNA fragments.

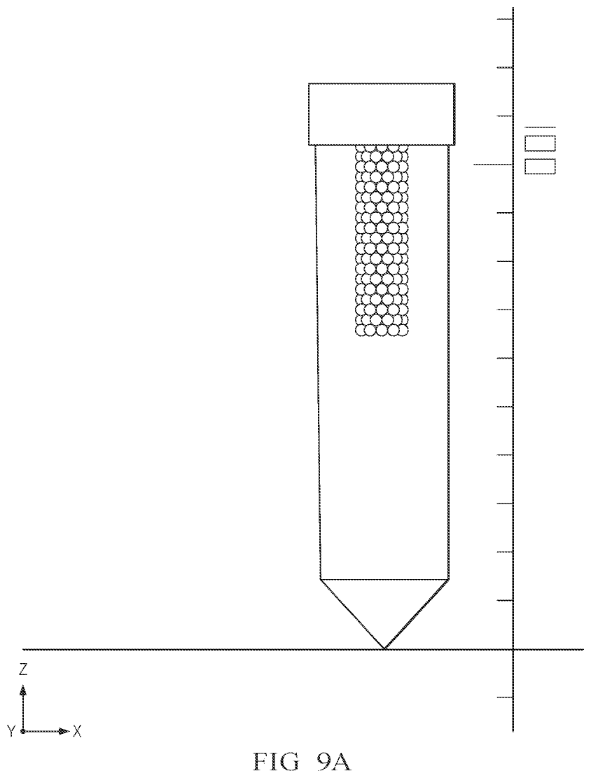

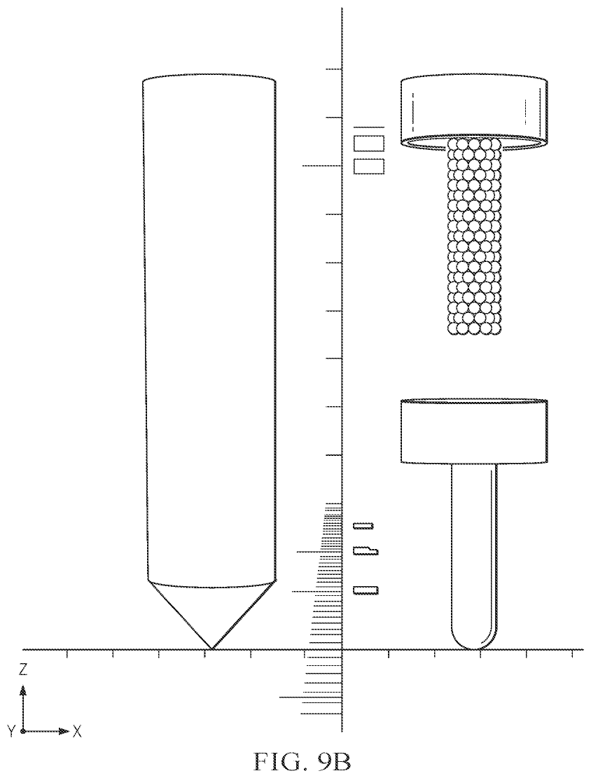

In view of the embodiments described above, in some embodiments, the present disclosure further pertains to, and can utilize, a dipstick that is configured to be in contact with a variety of free-standing solutions. FIG. 9A illustrates a conical tube with a dipstick (silica probed fixed to a cap). Biosamples, for example, fluid from a tampon or menstrual blood from a cup device can be transferred to a tube containing a DNA-binding buffer. cfDNA from the biosample will preferentially bind to the dipstick. After initial binding (e.g., >5 min) the dipstick can be transferred to an ethanol wash-containing tube and finally to a water-containing tube for sample elution. FIG. 9B illustrates that the dipstick can be composed of silica gel beads contained by a permeable mesh or as a solid rod of silica. While FIG. 9A and FIG. 9B illustrate an example of a contact surface in the form of a rod, it should be understood that any such contact surface is readily envisioned. For example, the systems and methods of the present disclosure can utilize a first contact surface, a second contact surface, or a first and second contact surface for applications such as, but not limited to, swabbing applications, “teabag” applications, or any other kind of device to contact an absorbent material, for example, silica beads with a biosample.

As illustrated above, various embodiments of the present disclosure can, among other uses, leverage the natural menstrual processes for completely at-home diagnostics (e.g., sexually transmitted diseases or infections). The various embodiments of the present disclosure are based, at least in part, on a number of hypotheses that include, without limitation, dynamic menses environments (e.g., natural cell lysis and/or cell turnover) will support release of cfDNA of the vaginal microbiome, cfDNA will be processed by a variety of extracellular restriction enzymes during menses in predictable ways (e.g., restriction enzymes (RE) also released due to lysis), and reprocessed cfDNA fragments are more accessible for targeting DNA strand-displacement circuits due to RE-produced single-stranded overhangs that can serve as toeholds (or “initiators”).

Menstrual DNA cfDNA isolation can occur at home, and cfDNA can be processed using only home-safe chemicals. As such, no centrifuges or harsh chemical lysis are involved. Menstrual cfDNA isolation can have a semi-optimized form, and similar to isolation, at home cfDNA processing uses only home-safe chemicals. In general, menstrual blood from tampons and/or cup devices are transferred to a tube containing a biomolecule processing buffer. Biomolecules from the biosample will preferentially bind the dipstick (FIG. 9A and FIG. 9B). Next, after waiting (generally, for example, >5 min) the dipstick is transferred to a wash solution. Finally, the dipstick is transferred for final biomolecule elution.

After menstrual DNA cfDNA isolation, characterization can begin (FIG. 5A and FIG. 5C). Generally, the characterization includes, without limitation, bioinformatic investigation of RE and cfDNA sequencing experiments. Next, pathogenic cfDNA target selection occurs (FIG. 6). Further, colorimetric detection of vaginal non-STD microflora from menstrual fluid can be determined.

Initial focus on restriction enzyme-based genome degradation was due to rationalization of the fact that fragmented DNA products are the prevalent extracellular DNA species in an unprocessed biosample. High-performance liquid chromatography (HPLC) analysis suggests that there is indeed some degradation and/or fragmentation occurring (resulting in a large population of DNA species as small as 100-200 bp. This collection of degraded DNA fragments (most relevant for diagnostic probing biosamples with minimal processing) has been named the degradome. The empirical findings indicate that there is degradation occurring. Furthermore, DNA sequencing can further reveal degradation patterns that can be used to better select diagnostic targets and design probes.

In some embodiments, the present disclosure pertains to a method of detecting specific sequences cfDNA from biological samples in real time and can include, without limitation, immersion of the biological samples into a receptacle (Receptacle 1) with a cap (e.g., a 50 mL conical tube), containing a solution, followed by a vigorous shaking to facilitate suspension resuspension of said biological samples in said solution. In some embodiments, the method further includes the removal of cap from Receptacle 1 and replacement of the cap with a dipstick cap, and shaking vigorously for a period of time. In some embodiments, the method further includes the removal of the dipstick cap from Receptacle 1 and placing the dipstick cap onto a second receptacle (Receptacle 2), containing a solution, and shaking vigorously for a period of time. In some embodiments, the method further includes the removal of the dipstick cap from Receptacle 2 and allowing the dipstick to air dry for a period of time. In some embodiments, the method further includes the placing the air-dried dipstick cap onto a third receptacle (Receptacle 3) containing a solution where cfDNA is eluted and resuspended. In some embodiments, the method then includes transferring the solution from Receptacle 3 to a reaction well where cfDNA is detected.

In some embodiments, the solution in Receptacle 1 is a solution that promotes adsorption of nucleic acids to silica. In some embodiments, the period of time to vigorously shake Receptacle 1 is about 30 seconds to 5 minutes. In some embodiments, the period of time to vigorously shake promote resuspension in Receptacle 1 with the dipstick is 30 seconds to 5 minutes. In some embodiments, the solution in Receptacle 2 is a washing solution, such as, for example, ethanol or other solvent that is similarly miscible in water and exhibits low nucleic acid solubility. In some embodiments, the period of time to vigorously shake Receptacle 2 is about 30 seconds to 5 minutes. In some embodiments, the period of time to allow the dipstick to air dry is about 30 seconds to 5 minutes. In some embodiments, the solution in Receptacle 3 is an aqueous solution that is more polar than the silica dipstick, such as, for example, water. In some embodiments, the period of time to wait for cfDNA to elute and resuspend is about 30 seconds to 10 minutes. In some embodiments, the solution containing cfDNA is transferred into reaction wells, vessels, or microfluidic chambers to permit detection of target cfDNA by complementary DNA oligos, or “DNA probes” partially or completely base-pairing to a specific cfDNA sequence unique to the target organism or specimen.

In another embodiment, the present disclosure pertains to a dipstick for capturing cfDNA from biological samples. In some embodiments, the dipstick includes a silica rod and/or tube and a cap connected to the rod and/or tube. In some embodiments, the rod and/or tube is composed of silica gel beads contained by a permeable mesh. In some embodiments, the rod and/or tube is a solid rod of silica. In some embodiments, the cap is a screw cap. In some embodiments, the rod has a length designed to fit within a 50 mL conical tube.

In a further embodiment, the present disclosure pertains to a method for at-home isolation of cfDNA from raw biological fluids. In general, the method includes: (1) addition of a raw biological fluid sample to an acidic, ionic formulation for a predetermined time; (2) filtration of clots and particulates from the corresponding solution using syringe filters; (3) binding of DNA within the filtered solution to a silica material via introduction of silica material; (4) washing the silica-adsorbed DNA to remove contaminants (e.g., proteins and/or lipids) from the silica material using, for example, ethanol; and (5) eluting DNA from the silica material, upon drying, by submerging the silica material in, for example, water.

In some embodiments, the predetermined time is shorter than 30 minutes. In some embodiments, the filters have a pore size greater than or equal to 0.22 μm. In some embodiments, the raw biological sample is menstrual fluid that is added in the form of a saturated tampon, sanitary napkin, or menstrual fluid poured from a menstrual cup. In some embodiments, the silica material is contained within a syringe membrane, and DNA is adsorbed to, washed, and eluted from the membrane via syringe filtering. In some embodiments, the silica material is composed of a solid silica rod for a teabag application. In some embodiments, the silica material is composed of silica beads contained inside of a netting, and surrounded by a wire mesh, for a teabag application. In some embodiments, the wire mesh is removed prior to washing steps. In some embodiments, the silica rod is attached to a lid for ease of transfer.

In another embodiment, the present disclosure pertains to a method for identifying optimal diagnostic TMSD probing sites for microbial detection in an at-home setting. In general, the method includes: (1) identification of degradome, or degradation patterns, of cfDNA of the targeted microbe; (2) compilation of a library of potential target toehold fragments, chosen as toeholds represented in the degradome; (3) design of a corresponding library of target sequences having sequence extension from each toehold to fill the design space of a predetermined length range for TMSD circuits; (4) ranking of the library of target sequences based on toehold abundance, length of target sequence, uniqueness of target sequence, and predicted energetics of interaction between target sequence and diagnostic DNA molecules.

In some embodiments, the degradome of the microbial cfDNA of interest is determined by activity nucleases of species, including and outside of the species of interest, represented in the biosample-corresponding microbiome. In some embodiments, degradome is identified computationally, via identification of endonucleases in the biosample of interest via homology-based alignment, and corresponding mapping of expected nuclease patterns. In some embodiments, the degradome is identified via experimental metagenomic DNA sequencing using specialized NGS preparation methods, where DNA size-selected fragments are not fragmented prior to sequencing.

In some embodiments, the degradome is compiled computationally and experimentally, such that standard probabilities of restriction enzyme activity to create unique toeholds are obtained from comparing mapped sequence positions corresponding to fragment ends to those identified in the bioinformatic approach. In some embodiments, only toehold lengths greater than 4 nucleotides are considered. In some embodiments, target sequences libraries are according to target sequence length between 25 and 40 nucleotides in length. In some embodiments, machine learning approaches are used to optimize the ranking of the library of target sequences.

In some embodiments, the present disclosure pertains to a system for visual detection of microbial cfDNA from raw biological fluids in the at-home setting, performed by a user. In some embodiments, the system includes: (1) a first solution acidic, ionic solution configured for mixing with raw biosample sample; (2) a silica-based material configured for extracting biomolecules from the solution; (3) a washing solution configured for washing the silica-based material of non-nucleic acid biomolecules; and (4) an elution solution composed of water and DNA diagnostic molecules enabling a TMSD cascade coupled to a positive visual output if presence of the diagnostic target is detected.

In some embodiments, targets are informed via a method to select optimal targets that incorporates, but is not limited to, degradome information, thermodynamics, and homology analysis. In some embodiments, TMSD optimization is achieved using, but not limited to, established DNA circuit-modeling software programs and logic multiplexing. In some embodiments, TMSD optimization is achieved by destabilizing dsDNA fragments containing the diagnostic DNA target sequence using a molar excess of oligonucleotides directed towards the surrounding sequence space. In some embodiments, colorimetric signal is enabled by color catalysis of a colorless substrate upon displacement-induced formation of a DNAzyme. In some embodiments, fluorometric signal is enabled by TMSD cascade-induced separation of fluorophore and quencher. In some embodiments, a distinct device for human sight-interpretation of fluorometric signal is included. In some embodiments, the raw biosample is menstrual fluid and the microbes to be detected are the causative agents of sexually-transmitted diseases and/or infections.

This disclosure contains the following sequences:

(SEQ ID NO: 01)
GCACTTTCGAGCATCTTCGGCCATTTCGCTATATCCTCCACGGAAATGGC
CGAAGATGCTCCTGATGTGGGCTAAAG
(SEQ ID NO: 02)
GCCATTTCCGTGGAGGATATAGCGAAATGGCCGAAGATGCTCGCTATATC
CTCCACG
(SEQ ID NO: 03)
CTCCTGATGTGGGCTAAAGTCCCTC
(SEQ ID NO: 04)
CTGGGAGGGAGGGAGGGACTTTAGCCCACATCAGGAGCATCTTCG
(SEQ ID NO: 05)
GAAATGGCCGAAGATGCTCGAAAGTGC
(SEQ ID NO: 06)
AGGTACGATACTACCGAAATGCAGA
(SEQ ID NO: 07)
TCCATGCTATGATGGCTTTACGTCT
(SEQ ID NO: 08)
TACTACCGAAATGC
(SEQ ID NO: 09)
CTTTACGTCT
(SEQ ID NO: 10)
CTTTACGTCTTCCATGCTATGATG
(SEQ ID NO: 11)
CTTTACGTCTTCCATGCTATGATGGA
(SEQ ID NO: 12)
CTTTACGTCTTCCATGCTATGATGGAGC

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

1-66. (canceled)

67. A method of detecting specific sequences of cell-free DNA (cfDNA) from a biological sample, the method comprising:
placing the biological sample into a first receptacle comprising a first solution;
wherein the first receptacle comprises a first contact surface;
facilitating resuspension of the biological sample in the first solution;
replacing the first contact surface with a second contact surface;
mixing the first solution containing the biological sample in the first receptacle to facilitate adsorption of extracellular DNA from the biological sample and the first solution;
removing the second contact surface from the first receptacle;
attaching the second contact surface to a second receptacle comprising a second solution;
wherein the second contact surface contacts at least a portion of a second solution in the second receptacle;
mixing the second solution in the second receptacle to facilitate saturation of the second contact surface with the second solution;
removing the second contact surface from the second receptacle;
placing at least a portion of the second solution onto a third receptacle comprising a third solution;
eluting and resuspending cfDNA from the biological sample to form a third solution;
transferring the third solution from the third receptacle to a reaction site;
wherein the third solution comprises the cfDNA from the biological sample; and
detecting the cfDNA from the biological sample at the reaction site.

68. The method of claim 67, wherein the first solution promotes adsorption of nucleic acids to an absorbent.

69. The method of claim 68, wherein the absorbent is selected from the group consisting of silica, sand, materials with similar absorbent properties of silica or sand, and combinations thereof.

70. The method of claim 67, wherein the mixing the first solution in the first receptacle is conducted over a period of time between 30 seconds to 5 minutes.

71. The method of claim 67, wherein the facilitating resuspension of the biological sample in the first solution is conducted over a period of time between 30 seconds to 5 minutes.

72. The method of claim 67, wherein the second solution comprises a washing solution.

73. The method of claim 72, wherein the washing solution is selected from the group consisting of ethanol and a solvent that is similarly miscible in water and exhibits a low nucleic acid solubility.

74. The method of claim 67, wherein the mixing the second solution in the second receptacle is conducted over a period of time between 30 seconds to 5 minutes.

75. The method of claim 67, wherein the second contact surface comprises a dipstick.

76. The method of claim 75, further comprising:
drying the dipstick; and
wherein the drying is conducted via air drying and over a period of time between 30 seconds to 5 minutes.

77. The method of claim 75, wherein the third solution an aqueous solution that is more polar than a coating on the dipstick.

78. The method of claim 77, wherein the coating is an absorbent.

79. The method of claim 78, wherein the absorbent is selected from the group consisting of silica, sand, materials with similar absorbent properties of silica or sand, and combinations thereof.

80. The method of claim 77, wherein the aqueous solution is water.

81. The method of claim 67, wherein the eluting and resuspending is conducted over a period of time between 30 seconds to 10 minutes.

82. The method of claim 67, wherein the third solution is transferred into at least one of reaction wells, vessels, or microfluidic chambers to permit detection of target cfDNA by at least one complementary DNA oligos or “DNA probes” partially or completely base-pairing to a specific cfDNA sequence unique to a target organism or specimen or gene.

83. The method of claim 67, wherein the third solution is transferred into at least one of reaction wells, vessels, or microfluidic chambers to permit detection of target cfDNA by at least one of polymerase chain reaction (PCR), loop-mediated isothermal amplification (LAMP), or antibody-based methods unique to a target organism or specimen or gene.


SEQUENCE LISTING (12 sequences)

SEQ ID NO: 1 - DNA, 77 bp, synthetic construct
  source 1..77: mol_type = other DNA
gcactttcgagcatcttcggccatttcgctatatcctccacggaaatggccgaagatgctcctgatgtgggctaaag

SEQ ID NO: 2 - DNA, 57 bp, synthetic construct
  source 1..57: mol_type = other DNA
gccatttccgtggaggatatagcgaaatggccgaagatgctcgctatatcctccacg

SEQ ID NO: 3 - DNA, 25 bp, synthetic construct
  source 1..25: mol_type = other DNA
ctcctgatgtgggctaaagtccctc

SEQ ID NO: 4 - DNA, 45 bp, synthetic construct
  source 1..45: mol_type = other DNA
ctgggagggagggagggactttagcccacatcaggagcatcttcg

SEQ ID NO: 5 - DNA, 27 bp, synthetic construct
  source 1..27: mol_type = other DNA
gaaatggccgaagatgctcgaaagtgc

SEQ ID NO: 6 - DNA, 25 bp, synthetic construct
  source 1..25: mol_type = other DNA
aggtacgatactaccgaaatgcaga

SEQ ID NO: 7 - DNA, 25 bp, synthetic construct
  source 1..25: mol_type = other DNA
tccatgctatgatggctttacgtct

SEQ ID NO: 8 - DNA, 14 bp, synthetic construct
  source 1..14: mol_type = other DNA
tactaccgaaatgc

SEQ ID NO: 9 - DNA, 10 bp, synthetic construct
  source 1..10: mol_type = other DNA
ctttacgtct

SEQ ID NO: 10 - DNA, 24 bp, synthetic construct
  source 1..24: mol_type = other DNA
ctttacgtcttccatgctatgatg

SEQ ID NO: 11 - DNA, 26 bp, synthetic construct
  source 1..26: mol_type = other DNA
ctttacgtcttccatgctatgatgga

SEQ ID NO: 12 - DNA, 28 bp, synthetic construct
  source 1..28: mol_type = other DNA
ctttacgtcttccatgctatgatggagc